(19) United States
(12) Patent Application Publication
(10) Pub. No.: US 2021/0123013 A1
(43) Pub. Date: Apr. 29, 2021

(54) SYSTEM FOR CELL CULTURE IN A BIOREACTOR

(71) Applicants: UNIVERSITE DE BORDEAUX, BORDEAUX (FR); CENTRE NATIONAL DE LA RECHERCHE SCIENTIFIQUE, PARIS (FR); INSTITUT D'OPTIQUE THÉORIQUE ET APPLIQUÉE, PALAISEAU CEDEX (FR)

(72) Inventors: MAXIME FEYEUX, TALENCE (FR); KEVIN ALESSANDRI, BORDEAUX (FR); PIERRE NASSOY, BORDEAUX (FR)

(21) Appl. No.: 17/057,143

(22) PCT Filed: May 20, 2019
(86) PCT No.: PCT/FR2019/051144

§ 371 Date: Nov. 20, 2020

(30) Foreign Application Priority Data
May 21, 2018 (FR) 1854207

(51) Int. Cl. C12N 5/00; C12M 1/32; C12M 1/00; C12N 1/04

(52) U.S. Cl. CPC C12N 5/0068; C12M 23/12; C12N 2506/00; C12N 1/04; C12N 2533/90; C12M 23/20

(57) ABSTRACT

The invention relates to a bioreactor cell culture system comprising a closed chamber containing a plurality of suspended cell microcompartments, wherein the microcompartments each comprise an outer hydrogel layer providing a cavity containing a set of self-organized cells and extracellular matrix or an extracellular matrix substitute. The invention further relates to the use of such bioreactors to produce cells and/or organoids, and/or molecules and/or complex molecular assemblies.

The invention relates to systems for cell culture in a bioreactor. The system according to the invention can be used for the production of cells of interest, of cell assemblies (organoids, tissues) and/or the production of molecules of interest, or of complex molecular assemblies (components of extracellular matrices, cell organelles, antibodies, vaccines, exosomes, viroids), or other materials of interest originating from cells or produced by cells grown in such systems.

Bioreactor cell culture systems are of increasing interest to the pharmaceutical industry, among others. Indeed, eukaryotic cells are increasingly used as a therapeutic tool, in particular in cell and tissue therapy, and as a tool for the bioproduction of molecules of interest, from protein fractions (insulin, antibodies, etc.), through complexes of proteins, lipids and sugars derived from cells or cell organelles, extracellular vesicles and exosomes, to viral derivatives (for the production of vaccines in particular). Bioreactor cell culture systems enable the mass cultivation of these cells and thus meet the needs for cells and/or molecules of interest on an industrial scale.

Currently, there are three main classes of bioreactor cell culture methods:
Methods allowing batch culture, in which cells are inoculated in a fixed volume of culture medium. After an adequate culture time to allow sufficient growth, the molecules and/or cells are harvested. The major problem with these methods is that the nutrients present in the medium are depleted over time, and toxic metabolites accumulate;
Methods allowing fed-batch culture, in which culture medium is added as needed to feed the cells while maintaining acceptable cell density. The main problem with these systems is that metabolic wastes are not removed and accumulate in the bioreactor, which ultimately affects yield;
Methods allowing perfusion culture, in which the culture medium is changed continuously to feed the cells and remove waste. Such systems allow a higher yield, but the rapid and continuous change of the culture medium requires to retain the cells without damaging them (mechanical stress generated by the flow).

In the prior art, these mass bioproduction methods have little or no applicability to fragile cells or fragile cell assemblies. Indeed, in suspension, in aggregate or on microcarriers, cells and cell assemblies are directly exposed in the culture medium to mechanical stresses (shock, shear stress, pressure, etc.). When volumes become large, the mechanical forces used to stir or circulate the medium can destroy the cells or cell assemblies, in particular by shear stress applied by liquid flows or impact with the moving elements that stir the medium.

### SUMMARY OF THE INVENTION

By working on these problems of cell culture in a bioreactor, the inventors discovered that it is possible to create a culture space within microcompartments delimited by an outer hydrogel layer to cultivate a large number of cells within a bioreactor. The cell niche of interest is thus surrounded by a hydrogel shell that advantageously allows nutrients to infiltrate and proteins and metabolites to exfiltrate but retains the elements whose size exceeds 150 kDa (extracellular matrix, exosomes, viral particles, cells). Moreover, since the cells are protected from the stresses that may exist within the reactor by the hydrogel shell, the flow through the bioreactor can be as strong as the hydrogel shell can support. Furthermore, the hydrogel shell of the cell microcompartments, unlike existing culture systems, protects the cells from mechanical stresses related to collisions and prevents fusions of the multicellular elements (aggregates, microcarriers) that exist during liquid suspension culture, which cause reproducibility problems by varying the local conditions experienced by the cells (diffusion distance in the medium, mechanical stresses). The microcompartments are suspended in the bioreactor, which allows homogeneous access to the culture medium and diffusion into the microcompartments, as well as good convection. In addition, since the cell niche is protected by the hydrogel shell, it is possible to cultivate the most fragile cell types under optimal yield conditions with low cell death and well-controlled phenotype. Unlike a simple spheroid encased in a gel, the cavity in the capsule leaves cells room to multiply and/or to self-organize on extracellular matrix. Advantageously, each microcompartment comprises a unique cell niche. In other words, a given hydrogel shell surrounds a single cell niche. Since the outer layer of the microcompartments is made of hydrogel, it can easily be dissolved to recover the cells at the end of production. Since these microcompartments are in 3D, they advantageously allow a cell amplification in the microcompartment by a factor of up to 100 000.

The invention thus has as its object a bioreactor cell culture system comprising a closed chamber containing a plurality of cell microcompartments, wherein the microcompartments each comprise an outer hydrogel layer providing a cavity containing a set of self-organized cells and extracellular matrix or an extracellular matrix substitute.

According to the invention, an outer hydrogel layer surrounds a set of cells. The hydrogel layer forms a hollow capsule, providing a cavity containing the set of cells.

Advantageously, the hydrogel capsule contains a unique set of cells.

According to the invention, the plurality of cell microcompartments is suspended in the bioreactor chamber. More particularly, the microcompartments float in the culture medium contained in the bioreactor chamber.

The invention also has as its object the use of such a bioreactor cell culture system, comprising a closed chamber, for the production and/or amplification of cells of interest. The amplification is advantageously by a factor of 2 to 100,000 between each passage. This amplification factor corresponds to the number of living cells harvested at the end of amplification, divided by the number of living cells inoculated.

The invention also has as its object the use of such a bioreactor cell culture system for the production of molecules of interest and/or complex molecular assemblies, such as components of extracellular matrices, cell organelles, antibodies, vaccines, exosomes, viroids, etc., said molecules and/or assemblies being excreted by the cells of the microcompartments out of said microcompartments into the culture medium, or conversely accumulated inside the microcompartment for subsequent harvest.

The invention also has as its object a process for the production of organoids or cells of interest comprising the steps according to which:
a plurality of cell microcompartments is introduced into a bioreactor, comprising a closed chamber, said microcompartments each comprising an outer hydrogel layer encapsulating cells and extracellular matrix or an extracellular matrix substitute;
the microcompartments are cultivated under conditions allowing the multiplication of cells within the microcompartments, and/or the self-organization of cells into organoids;
the cell microcompartments are recovered
and optionally, the hydrogel layer is hydrolyzed to recover the organoids or the cells of interest.

The invention also has as its object a process for the production of differentiated cells from multipotent, pluripotent or totipotent cells comprising the steps according to which:
a plurality of cell microcompartments is introduced into a bioreactor, said microcompartments each comprising an outer hydrogel layer encapsulating multipotent, pluripotent or totipotent cells and extracellular matrix or an extracellular matrix substitute;
the microcompartments are cultivated under conditions allowing the multiplication of cells within the microcompartments, and/or differentiation into one or more cell type(s) of interest;
the cell microcompartments are recovered
and optionally, the hydrogel layer is hydrolyzed to recover the cell type(s) of interest.

### DETAILED DESCRIPTION

The inventors discovered that it is possible and particularly advantageous to cultivate cells within a reactor comprising a closed chamber, by keeping the cells inside an outer capsule of crosslinked hydrogel. More precisely, the inventors developed cell microcompartments each comprising an outer hydrogel layer encapsulating a set of self-organized cells and extracellular matrix or an extracellular matrix substitute. According to the invention, the cell microcompartments are suspended in the bioreactor.

According to the invention, self-organized cells means a set of cells that are uniquely positioned relative to one another to create cellular interactions and communications and form a three-dimensional microstructure of interest. Each microcompartment thus comprises an outer hydrogel layer, or hydrogel capsule, containing a set of self-organized cells. Cells can multiply, organize and/or differentiate within the hydrogel capsule.

In an embodiment, the hydrogel capsule contains a unique set of self-organized cells. Unique means that the capsule contains only one group of cells, which may be more or less cohesive. In particular, a unique set of cells means a three-dimensional cell structure in which each cell of said set is in physical contact with at least one other cell of said set.

According to the invention, it is possible to encapsulate all kinds of eukaryotic cells, and more particularly mammalian cells. In particular, the cells are selected from differentiated cells, progenitors, stem cells, multipotent cells, pluripotent cells, totipotent cells, genetically modified cells, and mixtures thereof, etc. In an embodiment, the encapsulated cells are pluripotent stem cells, selected in particular from embryonic stem cells and/or induced pluripotent cells (IPS). In an embodiment, the encapsulated cells are embryonic stem cells, in particular pluripotent embryonic stem cells. In an embodiment, the encapsulated cells are embryonic stem cells, excluding human embryonic stem cells having required the destruction of a human embryo. In another embodiment, the encapsulated cells are human embryonic stem cells derived from supernumerary human embryos conceived in the context of medically assisted procreation that is no longer the subject of a parental project, in accordance with the bioethical laws in force at the time and in the country where said embryonic stem cells were obtained. In another embodiment, the encapsulated cells are induced pluripotent cells (IPS), and in particular human induced pluripotent cells (hIPS). In another embodiment, the encapsulated cells are embryonic stem cells and induced pluripotent cells. In an embodiment, the encapsulated cells comprise a mixture of embryonic stem cells and induced pluripotent cells.

In the context of the invention, “outer hydrogel layer” or “hydrogel shell” denotes a three-dimensional structure formed from a matrix of polymer chains swollen by a liquid, and preferentially water. Such an outer hydrogel layer is obtained by crosslinking a hydrogel solution. Advantageously, the polymer(s) of the hydrogel solution are crosslinkable polymers when subjected to a stimulus such as temperature, pH, ions, etc. Advantageously, the hydrogel solution used is biocompatible, in the sense that it is not toxic to cells. The hydrogel layer advantageously allows the diffusion of dissolved gases (and in particular oxygen and/or carbon dioxide), nutrients, and metabolic wastes to allow the survival, proliferation, differentiation, maturation of cells and/or the production of molecules or molecular assemblies of interest and/or the recapitulation of cellular behaviors of interest. The polymers of the hydrogel solution can be of natural or synthetic origin. For example, the hydrogel solution contains one or more polymers among sulfonate-based polymers, such as sodium polystyrene sulfonate, acrylate-based polymers, such as sodium polyacrylate, polyethylene glycol diacrylate, the gelatin methacrylate compound, polysaccharides, and in particular polysaccharides of bacterial origin, such as gellan gum, or of plant origin, such as pectin or alginate. In an embodiment, the hydrogel solution contains at least alginate. Preferentially, the hydrogel solution contains only alginate. In the context of the invention, “alginate” means linear polysaccharides formed from β-D-mannuronate (M) and α-L-guluronate (G), salts and derivatives thereof. Advantageously, the alginate is a sodium alginate, composed of more than 80% G and less than 20% M, with an average molecular mass of 100 to 400 kDa (for example: PRONOVA® SLG100) and a total concentration comprised between 0.5% and 5% by density (weight/volume).

According to the invention, the cell microcompartment is closed. It is the outer hydrogel layer that gives the cell microcompartment its size and shape. The microcompartment can have any shape compatible with the encapsulation of cells.

Preferentially, the extracellular matrix layer forms a gel. The extracellular matrix layer comprises a mixture of proteins and extracellular compounds necessary for cell culture, for example pluripotent cells. Preferentially, the extracellular matrix comprises structural proteins, such as laminin 521, 511 or 421, entactin, vitronectin, laminins, collagen, as well as growth factors, such as TGF-beta and/or EGF. In an embodiment, the extracellular matrix layer consists of or contains Matrigel® and/or Geltrex®.

According to the invention, the microcompartment may contain, in place of the extracellular matrix, an extracellular matrix substitute. An extracellular matrix substitute means a compound capable of promoting cell attachment and/or survival by interacting with membrane proteins and/or extracellular signal transduction pathways. For example, such a substitute comprises biological polymers and fragments thereof including proteins (laminins, vitronectins, fibronectins and collagens), nonsulfated glycosaminoglycans (hyaluronic acid) or sulfated glycosaminoglycans (chondroitin sulfate, dermatan sulfate, keratan sulfate, heparan sulfate), and synthetic polymers containing units derived from biological polymers or reproducing their properties (RGD unit) and small molecules mimicking attachment to a substrate (Rho-A kinase inhibitors such as Y-27632 or thiazovivin).

Any method for the production of cell microcompartments containing extracellular matrix and cells within a hydrogel capsule may be used for carrying out the preparation process according to the invention. In particular, it is possible to prepare microcompartments by adapting the method and the microfluidic device described in Alessandri et al. 2016 (“A 3D printed microfluidic device for production of functionalized hydrogel microcapsules for culture and differentiation of human Neuronal Stem Cells (hNSC)”, Lab on a Chip, 2016, vol. 16, no. 9, pp. 1593-1604).

Advantageously, the dimensions of the cell microcompartment are controlled. In an embodiment, the cell microcompartment according to the invention has a spherical shape. Preferentially, the diameter of such a microcompartment is comprised between 10 μm and 1 mm, more preferentially between 50 μm and 500 μm, even more preferentially less than 500 μm, preferably less than 400 μm. In another embodiment, the cell microcompartment according to the invention has an elongated shape. In particular, the microcompartment may have an ovoid or tubular shape. Advantageously, the smallest dimension of such an ovoid or tubular microcompartment is comprised between 10 μm and 1 mm, more preferentially between 50 μm and 500 μm, even more preferentially less than 500 μm, preferably less than 400 μm. “Smallest dimension” means twice the minimum distance between a point located on the outer surface of the hydrogel layer and the center of the microcompartment.

In a particular embodiment, the thickness of the outer hydrogel layer represents 5 to 40% of the radius of the microcompartment. The thickness of the extracellular matrix layer represents 5 to 80% of the radius of the microcompartment and is advantageously hung on the inner face of the hydrogel shell. This matrix layer can fill the space between the cells and the hydrogel shell. In the context of the invention, the “thickness” of a layer is the dimension of said layer extending radially from the center of the microcompartment.

In an embodiment of the invention, the bioreactor comprises microcompartments in which the cells are self-organized into cysts.

In the context of the invention, a cyst is defined as at least one layer of pluripotent or totipotent cells organized around a central lumen. According to the invention, such a microcompartment thus comprises successively, around a central lumen, said layer of pluripotent cells, a layer of extracellular matrix, or of an extracellular matrix substitute, and the outer hydrogel layer. The lumen is generated, at the moment of cyst formation, by the cells which multiply and develop in layers on the extracellular matrix layer. Advantageously, the lumen contains a liquid and more particularly culture medium.

According to the invention, a cyst advantageously contains one or more layers of pluripotent stem cells of a mammal, human or nonhuman. A pluripotent stem cell, or pluripotent cell, means a cell that has the capacity to form all tissues present in the whole organism of origin, without being able to form a whole organism as such. In particular, a cyst may contain embryonic stem cells (ESC), induced pluripotent stem (IPS) cells, or multilineage-differentiating stress enduring (MUSE) cells found in adult mammalian skin and bone marrow.

Advantageously, the thickness of the outer hydrogel layer represents 5 to 40% of the radius of the microcompartment, the thickness of the extracellular matrix layer represents 5 to 80% of the radius of the microcompartment and the thickness of the pluripotent cell layer represents about 10% of the radius of the microcompartment. The pluripotent cell layer is in contact at least at one point with the extracellular matrix layer, a space filled with culture medium may be present between the matrix layer and the cyst. The lumen then represents 5 to 30% of the radius of the microcompartment. In a particular example, the cell microcompartment has a spherical shape with a radius equal to 100 μm. The hydrogel layer has a thickness of 5μm to 40 μm. The extracellular matrix layer has a thickness of 5 μm to about 80 μm. The pluripotent cell layer has a thickness of 10 to 30 μm, the lumen having a radius of 5 to 30 μm, roughly.

According to an example embodiment of the invention, it is possible to cultivate in a bioreactor for example of 150 mL such microcompartments, in which the cells form cysts, according to the steps below:
(a) Incubate 600,000 to 2 million mammalian pluripotent stem cells in culture medium containing an inhibitor of the RHO/ROCK pathways;
(b) mix these pluripotent stem cells derived from step (a) with an extracellular matrix;
(c) encapsulate the mixture from step (b) in a hydrogel layer;
(d) cultivate the capsules obtained in step (c) in a culture medium containing an inhibitor of the RHO/ROCK pathways;
(e) rinse the capsules derived from step (d), so as to remove the inhibitor of the RHO/ROCK pathways;

(f) cultivate in a fed-batch type production mode the capsules derived from step (e) for 3 to 20 days, preferentially 5 to 10 days, by diluting the volume of medium by a factor of two each day with a pluripotent cell culture medium such as MTESR1 (Stemcell Technologies) free of inhibitors of the RHO/ROCK pathways, and optionally recover the cell microcompartments obtained.

The person skilled in the art will know how to adapt the number of cells and the volume of the bioreactor according to needs.

Step (a) of incubation and step (d) of culture in a medium containing one or more inhibitors of the RHO/ROCK (“Rho-associated protein kinase”) pathways, such as thiazovivin (C₁₅H₁₃N₅OS) and/or Y-27632 (C₁₄H₂₁N₃O), promote the survival of pluripotent stem cells and the adhesion of the cells to the extracellular matrix at the moment of formation of the outer hydrogel layer around said extracellular matrix. It is however desirable that these steps be limited in time, so that the inhibitors of the RHO/ROCK pathways do not prevent the formation of cysts.

Thus, preferentially, the incubation of step (a) is conducted for a period of time comprised between a few minutes and a few hours, preferentially between 2 minutes and 2 hours, more preferentially between 10 minutes and 1 hour.

Similarly, preferentially, the culture step (d) is conducted for a period of time comprised between 2 and 48 hours, preferentially for a period of time between 6 and 24 hours, more preferentially for a period of time between 12 and 18 hours.

Step (e) is necessary to ensure the removal of any trace of inhibitors of the RHO/ROCK pathways. Step (e) is for example performed by rinsing, and preferentially several rinses, in successive culture media free of inhibitors of the RHO/ROCK pathways.

Advantageously, step (f) is conducted for a sufficient time to obtain a cell microcompartment in which the layers of extracellular matrix and pluripotent cells have a cumulative thickness equal to 50 to 100% of the thickness of the outer hydrogel layer. Any culture medium suitable for the cultivation of pluripotent stem cells may be used.

In an embodiment, the process according to the invention comprises an intermediate step (a′) consisting in dissociating the pluripotent stem cells derived from step (a) before step (b), preferentially by means of an enzyme-free reagent. Advantageously, said reagent is inhibited or rinsed before the encapsulation step, in particular by successive rinsing in a specific medium for pluripotent cells. For example, the reagent used is ReLeSR®. Of course, it is also possible to use trypsin or a reagent containing an enzyme, but the survival rate of the pluripotent cells after this step may then be lower compared with the use of an enzyme-free reagent.

Alternatively, such microcompartments can be obtained according to the steps below:
(A) mix mammalian differentiated cells with an extracellular matrix and cell reprogramming agents;
(B) encapsulate the mixture from step (A) in a hydrogel layer;
(C) cultivate the capsules derived from step (B) for at least 3 days, and optionally recover the cell microcompartments obtained.

For example, the differentiated cells used are fibroblasts, peripheral blood mononuclear cells, epithelial cells and more generally cells derived from liquid or solid biopsies of human tissues.

The skilled person knows how to reprogram a differentiated cell into a stem cell by reactivating the expression of genes associated with the embryonic stage by means of specific factors. By way of examples, mention may be made of the methods described in Takahashi et al., 2006 (“Induction of pluripotent stem cells from mouse embryonic and adult fibroblast cultures by defined factors” Cell, 2006 Vol 126, pages 663-676) and in the international application WO2010/105311 entitled “Production of reprogrammed pluripotent cells”.

The reprogramming agents are advantageously co-encapsulated with the differentiated cells, so as to concentrate the product and promote contact with the set of cells.

Reprogramming agents make it possible to impose on the cells a succession of phenotypic changes up to the pluripotent stage. Advantageously, the reprogramming step (A) is performed using specific culture media, promoting these phenotypic changes. For example, the cells are cultured in a first medium comprising 10% human or bovine serum in a minimum essential Eagle medium (DMEM) supplemented with a serine/threonine protein kinase receptor inhibitor (such as the product SB-431542 (C₂₂H₁₆N₄O₃)), one or more inhibitors of the RHO/ROCK (“Rho-associated protein kinase”) pathways, such as thiazovivin and/or Y-27632, fibroblast growth factors, such as FGF-2, ascorbic acid and antibiotics, such as Trichostatin A (C₁₇H₂₂N₂O₃). The culture medium is then replaced by a medium promoting the multiplication of pluripotent cells, such as the medium mTeSR®1.

Such cysts can then be forced into a differentiation pathway of interest, so as to obtain microcompartments containing one or more cell types of interest, in particular for the production of molecules of interest, or the production of organoids of interest.

In an embodiment, the bioreactor comprises microcompartments comprising cells self-organized into organoids.

In the context of the invention, an organoid is defined as a multicellular structure organized in three dimensions so as to reproduce the microstructure of at least part of an organ. According to the invention, such a microcompartment thus comprises a three-dimensional multicellular structure, surrounded by extracellular matrix, the whole being encapsulated in the outer hydrogel layer.

According to the invention, the organoids can be obtained by encapsulating pluripotent or progenitor cells that are then differentiated within the hydrogel capsule, or by directly encapsulating differentiated or mature cells.

In an embodiment, the cell microcompartments introduced into the bioreactor contain pluripotent cells. A step of cell differentiation into at least one cell type of interest is then performed inside the bioreactor, and optionally a step of multiplication of said differentiated cells in the microcompartments.

In an embodiment, the cell microcompartments introduced into the bioreactor contain already differentiated cells or progenitors. A step of multiplication and/or maturation of said differentiated cells in the microcompartments is then performed inside the bioreactor.

Advantageously, the microcompartments introduced into the bioreactor have an initial cell density of less than 10% occupancy of the internal volume of the microcompartments, preferentially less than 1%, even more preferentially less than 0.1%.

Advantageously, the microcompartments recovered at the end of the culture step in the bioreactor have a cell density greater than 10% occupancy of the internal volume of the microcompartments.

According to the invention, the cells contained in the hydrogel capsules are subjected to the flow of medium contained in the bioreactor and which passes through the hydrogel layer.

Advantageously, the ratio of convective volume outside the microcompartments to diffusive volume inside the microcompartments is comprised between 1 and 10,000, preferentially between 1 and 1000, more preferentially between 1 and 100.

According to the invention, the convective volume denotes the volume of culture medium inside the reactor chamber, between the microcompartments. The microcompartments being suspended in the bioreactor, the convective volume thus represents the medium circulating between the microcompartments. Conversely, the diffusive volume denotes the volume of culture medium diffusing inside the microcompartments, i.e. in the space(s)/void(s) created around/between/by the cells once self-organized.

Thus, in the case of a microcompartment containing a cyst, the diffusive volume is chiefly constituted by the central lumen and, at the beginning of the growth of the cyst, the space between the capsule wall and the cyst. In the case of a microcompartment containing an organoid, the diffusive volume consists chiefly of the spaces created within the three-dimensional multicellular structure.

The microcompartments according to the invention are advantageously characterized by the presence within the hydrogel capsule of one or more lumens, or one or more spaces, free of cells and allowing exactly the multiplication or self-organization of the cells inside the microcompartment. Skilled persons will know how to harvest the cells at the most adequate moment for their amplification or differentiation process corresponding to a certain level of saturation of the optimal space in this context.

In an embodiment, the microcompartments occupy between 0.01% and 74% of the volume of the bioreactor chamber.

The use of cell microcompartments makes it possible to cultivate cells in any type of bioreactor, equipped with a closed chamber, and in particular in a bioreactor in batch, fed-batch or continuous feed (perfusion) modes. The use of these microcompartments is particularly advantageous in the case of continuous feed culture. Indeed, the cells being protected by the hydrogel shell, it is possible to subject them to continuous flows without risk of weakening them.

In an embodiment, the bioreactor comprises a chamber that can be sealed hermetically. This makes it possible to control the atmosphere inside the bioreactor, and for example to cultivate the microcompartments under inert atmosphere.

The cell culture system according to the invention may comprise a chamber having a volume comprised between 1 mL and 10,000 L, preferentially between 5 mL and 10,000 L, between 10 mL and 10,000 L, between 100 mL and 10,000 L, between 200 mL and 10,000 L, between 500 mL and 10,000 L. In an embodiment, the chamber has a volume of at least 1 mL. In an embodiment, the chamber has a volume of at least 10 mL. In an embodiment, the chamber has a volume of at least 100 mL. In an embodiment, the chamber has a volume of at least 500 mL.

In an embodiment, the chamber has a volume of at least 1 L. In an embodiment, the chamber has a volume of at least 10 L. In an embodiment, the chamber has a volume of 100 L or more. Advantageously, any bioreactor comprising a closed chamber and capable of industrial-scale production of cells, organoids, molecules and/or complex molecular assemblies can be used.

In general, the use of a closed chamber allows a fine control of the culture environment, without risk of disturbance by the external environment. Furthermore, it is easy to obtain sterile products. It also allows a better volumetric yield.

In an embodiment, the microcompartments comprise between 10% and 98% by volume of cells at harvest, i.e. between 100 and 1,000,000 of cells depending on the diameter of the compartment concerned and the size of the cells produced, which can be calculated by the ratio between the total number of cells produced (as measured by the skilled person with a Malassez cell or an automated cell counter) and the number of capsules obtained (as measured by the skilled person by characterizing the volume of capsules by manual counting under an optical microscope or by automated image analysis). Of course, it is possible to begin cell culture with microcompartments comprising a smaller number of cells at the start, and in particular between 1 and 1,000 cells, i.e. 0.01% and 10% by volume occupied by the cells within the microcompartment depending on the diameter of the compartment concerned and the size of the cells produced. More generally, the microcompartments according to the invention comprise between 0.01% and 98% by volume of cells.

The cells can then multiply inside the microcompartment and self-organize, in particular into organoids.

In an embodiment, the cells of a microcompartment are all of the same cell type. According to the invention, the cells of the same microcompartment are all considered to be of the same cell type if at least 50%, preferentially 70%, more preferentially 90%, even more preferentially 98% or more of the cells of said microcompartment have the same phenotype, according to the knowledge of the person skilled in the art making it possible to characterize this cell type. In another embodiment, the cells of a microcompartment are of at least two different cell types. Advantageously, between 20 and 100% of the cells of a compartment have the same phenotype.

According to the invention, it is possible to cultivate within the same bioreactor microcompartments all comprising the same cell types, or conversely having different cell types. For example, the bioreactor may contain two types of microcompartments, each containing a particular cell type.

The culture system according to the invention is particularly advantageous for the production and/or amplification of cells of interest. Indeed, the organization of the cells within the hydrogel capsule, together with the extracellular matrix, allows their multiplication by a factor of 2 to 100,000 between each passage.

Passage means the manipulation of cells to add space or culture surface in order to continue amplification or to initiate differentiation or self-organization into organoids. This operation may make it necessary, in the example of microcarriers, to reload the bioreactor with new microcarriers. For the standard two-dimensional culture of adherent, pluripotent stem cells, this operation consists in detaching the cells from the old culture medium in order to reinoculate a new culture medium with greater surface area; for the skilled person, this operation may result in the loss of 50% of the cells. For culture in microcompartments according to the invention, this corresponds to the dissociation of the microcompartments, the dissociation of the self-organized cell sets or their dispersion into cell sets small enough to be encapsulated again in new microcompartments.

The invention has in particular as its object the use of such a bioreactor cell culture system for the mass production of pluripotent cells.

The invention also has as its object the use of such a bioreactor cell culture system for the production of unipotent or multipotent progenitors from pluripotent cells.

The invention also has as its object the use of such a bioreactor cell culture system for the production of terminally differentiated cells (i.e. corresponding to one or more specific functions) from pluripotent cells and/or unipotent or multipotent progenitors and/or combinatorials of these progenitors.

The invention has in particular as its object a process for the production of organoids or cells of interest comprising the steps according to which:
a plurality of cell microcompartments is introduced into a bioreactor comprising a closed chamber, said microcompartments each comprising an outer hydrogel layer encapsulating cells and extracellular matrix or an extracellular matrix substitute;
the microcompartments are cultivated under conditions allowing the multiplication of cells within the microcompartments, and/or the self-organization of cells into organoids;
the cell microcompartments are recovered
and optionally, the hydrogel layer is hydrolyzed to recover the organoids or the cells of interest.

The skilled person is able to adapt the culture conditions to the cell type of the microcompartments, in order to promote their multiplication and/or self-organization.

In an embodiment, the cell microcompartments introduced contain pluripotent cells, said process comprising, inside the bioreactor, a step of cell differentiation into at least one cell type of interest and a step of multiplication of said differentiated cells in the microcompartments. For example, the production of primitive endoderm organoids for the study of differentiation in human endodermic tissues can be carried out according to the following protocol:
From step f) of obtaining microcompartments, described above, at 2-3 days of culture:
Culture in a 150 mL closed bioreactor in a STEMdiff™ Pancreatic stage 1 medium of the STEMdiff™ Pancreatic Progenitor Kit marketed by STEMCELL Technologies for 3 to 6 days.
Use of the primitive endoderm obtained for developmental studies.

In another embodiment, the cell microcompartments introduced contain already differentiated cells or progenitors, said process comprising, inside the bioreactor, a step of multiplication of said differentiated cells in the microcompartments.

During the multiplication and/or maturation step, the cells will advantageously self-organize into a specific organoid, according to an organization specific to said cell type.

In an embodiment concerning amplification, the microcompartments introduced into the bioreactor have a cell density of less than 10% occupancy of the internal volume of the microcompartments, preferentially 1%, even more preferentially 0.1%. The cells will then multiply inside the microcompartments, during the culture step.

In an embodiment concerning differentiation and/or maturation without amplification, the microcompartments introduced in the bioreactor have a cell density higher than 1% occupancy of the internal volume of the microcompartments. The cells will then differentiate and/or mature and/or self-organize inside the microcompartments, during the culture step. For example, a first type of production of neural organoids for neuronal transplantation in the context of cell therapy for Parkinson's disease was carried out according to the following protocol:
Thawing of 5 million dopaminergic progenitors such as those marketed by Cellular Dynamics International (iCell® DopaNeurons),
Encapsulation of pre-differentiated neural progenitors according to the protocol described in Alessandri et al. 2016.
Culture in a 150 mL closed bioreactor in the culture medium provided by Cellular Dynamics.
Maturation and structuring of dopaminergic neural organoids for two weeks within the bioreactor.
Preparation of the transplant by dissociation of the hydrogel capsule by means of two 30-second rinses in 1 mL of ReLeSR® (Stemcell Technologies) then resuspension in a solution of 11% by mass of 70 kDa dextran in the neuron culture medium, distribution in a glass cannula of our own manufacture.
Transplant in an animal model of Parkinson's disease.

In an embodiment combining amplification and differentiation/maturation, the microcompartments introduced into the bioreactor advantageously have a cell density of less than 10% occupancy of the internal volume of the microcompartments, preferentially 1%, even more preferentially 0.1%. The cells will then multiply inside the microcompartments, during the culture step and then during the differentiation step. The cells will then self-organize inside the microcompartments, during a second culture step which can be triggered by a change in the nature of the nutrient medium or a physical trigger (temperature, illumination). For example, a second type of neural organoid production for neuron transplantation in the context of cell therapy for Parkinson's disease was carried out according to the following protocol:
From step f) of obtaining microcompartments described above at 2-3 days of culture:
Culture in a 150 mL closed bioreactor in a neural induction medium containing inhibitors of the BMP2 (2 μm dorsomorphin or 0.5 μm LDN 193189) and TGFbeta (10 μm+SB 431542) signaling pathways, 10 μm 24(S),25-epoxycholesterol on a neurobasal/DMEM-F12 base supplemented with N2 and B27 for 1 to 2 days.
Culture in a 150 mL closed bioreactor in a neural regionalization medium containing inhibitors of the BMP2 (2 μm dorsomorphin or 0.5 μm LDN 193189) and TGFbeta (10 μm +SB 431542) signaling pathways, two activators of the SHH pathway (200 ng/mL SHH; 1 μm purmorphamine) and FGF8 (100 ng/mL), an inhibitor of the WNT pathway (3 μm Chir99021), 10 μm 24(S),25-epoxycholesterol on a neurobasal/DMEM-F12 base supplemented with N2 and B27 for 6 days.
Culture in a 150 mL closed bioreactor in a second neural regionalization medium containing an inhibitor of the BMP2 signaling pathway (2 μm dorsomorphin or 0.5 μm LDN 193189), an inhibitor of the WNT pathway (3 μm Chir99021), 10 μM 24(S),25-epoxycholesterol on neurobasal/DMEM-F12 base supplemented with N2 and B27 for 1 day.
Culture in a 150 mL closed bioreactor in a medium for maturation and structuring of dopaminergic neural organoids for two weeks in the bioreactor containing cyclic AMP (500+ascorbic acid (200 μM)+GDNF (20 ng/mL)+BDNF (20 ng/mL)+FGF-20 (5 ng/mL)+TGFbeta (1 ng/mL)+trichostatin (10 nM)+Compound E (1 μM).
Preparation of the transplant by dissociation of the hydrogel capsule by means of two thirty-second rinses in 1 mL of ReLeSR® (Stemcell Technologies) then resuspension in a solution of 11% by mass of 70 kDa dextran in the neuron culture medium, distribution in a glass cannula of our own manufacture.
Transplant in an animal model of Parkinson's disease.

In another embodiment combining amplification and differentiation/maturation, the microcompartments introduced into the bioreactor advantageously have a cell density of less than 10% occupancy of the internal volume of the microcompartments, preferentially 1%, even more preferentially 0.1%. The cells will then multiply inside the microcompartments.

The cells are then recovered by dissolution of the capsule, then subjected to a second encapsulation step followed by the differentiation step, the cells will then self-organize inside the microcompartments, during a second culture step which can be triggered by a change in the nature of the nutrient medium or a physical trigger (temperature, illumination). For example, the production of human pancreatic organoids for human pancreatic tissue transplantation was carried out according to the following protocol:
From step f) of obtaining microcompartments described above at 2-3 days of culture:
Culture in a 150 mL closed bioreactor in a STEMdiff™ Pancreatic stage 1 medium supplemented with supplement 1A and supplement 1B of the STEMdiff™ Pancreatic Progenitor Kit marketed by STEMCELL Technologies for 1 day.
Culture in a 150 mL closed bioreactor in a STEMdiff™ Pancreatic stage 1 medium supplemented with supplement 1B of the STEMdiff™ Pancreatic Progenitor Kit marketed by STEMCELL Technologies for 1 day.
Culture in a 150 mL closed bioreactor in a STEMdiff™ Pancreatic stage 2-4 medium supplemented with supplement 2A and supplement 2B of the STEMdiff™ Pancreatic Progenitor Kit marketed by STEMCELL Technologies for 1 day.
Culture in a 150 mL closed bioreactor in a STEMdiff™ Pancreatic stage 2-4 medium supplemented with supplement 2A and supplement 2B of the STEMdiff™ Pancreatic Progenitor Kit marketed by STEMCELL Technologies for 2 days.
Culture in a 150 mL closed bioreactor in a STEMdiff™ Pancreatic stage 2-4 medium complemented with supplement 3 of the STEMdiff™ Pancreatic Progenitor Kit marketed by STEMCELL Technologies for 3 days.
Culture in a 150 mL closed bioreactor in a STEMdiff™ Pancreatic stage 2-4 medium complemented with supplement 3 of the STEMdiff™ Pancreatic Progenitor Kit marketed by STEMCELL Technologies for 5 days.
Preparation of the transplant by dissociation of the hydrogel capsule by means of two thirty-second rinses in 1 mL of ReLeSR® (Stemcell Technologies) then resuspension in a solution of 11% by mass of 70 kDa dextran in the previous medium, distribution in a glass cannula of our own manufacture.
Transplant in an animal model of type 1 diabetes.

Advantageously, the microcompartments recovered at the end of the culture step in the bioreactor have a cell density greater than 10% occupancy of the internal volume of the microcompartments, preferentially greater than 50%, and which can go in the case of organoids up to 98% occupancy.

The culture system according to the invention is also particularly attractive for the production of molecules of interest and/or complex molecular assemblies, said molecules and/or complex molecular assemblies being excreted by the cells of the microcompartments out of said microcompartments into the culture medium, or conversely accumulated inside the microcompartment for subsequent harvesting. This production method makes it possible in particular to limit the filtration steps of the cellular elements by concentrating them inside the microcompartments. This method allows, by virtue of the separation in the bioreactor of the convective and diffusive volumes by the capsule, an easier segregation of the medium containing the dissolved elements from the elements which are insoluble or larger than the mesh size of the hydrogel of the capsule (typically 150 to 250 kDa for alginate).

According to the invention, the microcompartments are then advantageously used in a reactor in continuous feed mode. As explained above, the presence of the protective hydrogel shell makes it possible to perfuse the culture medium at a flow rate without the risk of damaging the cells. In particular, it is possible to perfuse the inside of the reactor with culture medium at a flow rate comprised between 0.001 and 100 volumes of cells contained in the bioreactor per day.

1-16. (canceled)

17. A bioreactor cell culture system comprising a closed chamber containing a plurality of suspended cell microcompartments, wherein the microcompartments each comprise an outer hydrogel layer providing a cavity containing a set of self-organized cells and extracellular matrix or an extracellular matrix substitute.

18. The bioreactor cell culture system as claimed in claim 17, wherein the thickness of the outer hydrogel layer represents 5 to 40% of the radius of the microcompartment.

19. The bioreactor cell culture system as claimed in claim 17, wherein the thickness of the extracellular matrix layer or extracellular matrix substitute represents 5 to 80% of the radius of the microcompartment.

20. The bioreactor cell culture system as claimed in claim 17, wherein the thickness of the outer hydrogel layer represents 5 to 40% of the radius of the microcompartments, the thickness of the extracellular matrix layer or extracellular matrix substitute represents 5 to 80% of the radius of the microcompartments and the thickness of the pluripotent cell layer represents about 10% of the radius of the microcompartments.

21. The bioreactor cell culture system as claimed in claim 17, wherein the cell microcompartments have a spherical shape, wherein a diameter of said microcompartments is between 10 μm and 1 mm.

22. The bioreactor cell culture system as claimed in claim 17, wherein the cell microcompartments have an ovoid or tubular shape, wherein a smallest dimension of said an ovoid or tubular microcompartments is between 10 μm and 1 mm.

23). The bioreactor cell culture system as claimed in claim 17, wherein the ratio of convective volume outside the microcompartments to diffusive volume inside the microcompartments is between 1 and 10000.

24. The bioreactor cell culture system as claimed in claim 17, wherein all or part of the microcompartments comprise cells self-organized into cysts.

25. The bioreactor cell culture system as claimed in claim 17, wherein all or part of the microcompartments comprise cells self-organized into organoids.

26. The bioreactor cell culture system as claimed in claim 17, wherein the self-organized cells are selected from progenitors, stem cells, pluripotent cells, and mixture thereof.

27. The bioreactor cell culture system as claimed in claim 17, wherein the self-organized cells are selected from pluripotent stem cells from a human mammal, progenitors thereof, or mixture thereof

28. The bioreactor cell culture system as claimed in claim 17, wherein the bioreactor is selected from batch mode bioreactors, fed-batch mode bioreactors and continuous mode bioreactors.

29. The bioreactor cell culture system as claimed in claim 17, wherein the chamber has a volume between 1 mL and 10,000 L.

30. The bioreactor cell culture system as claimed in claim 17, wherein the microcompartments contain between 0.01% and 98% by volume of cells.

31. The bioreactor cell culture system as claimed in claim 17, wherein the cells of a microcompartment are all of the same cell type.

32. The bioreactor cell culture system as claimed in claim 17, wherein the cells of a microcompartment are of at least two different cell types.

33. The bioreactor cell culture system as claimed in claim 17, wherein the microcompartments all comprise the same cell types.

34. The bioreactor cell culture system as claimed in claim 17, wherein the microcompartments have at least partially different cell types.

35. A process for the production of organoids or cells of interest comprising the steps according to which:
a plurality of cell microcompartments is introduced into a bioreactor, said microcompartments each comprising an outer hydrogel layer encapsulating cells and extracellular matrix or an extracellular matrix substitute;
the microcompartments are cultivated under conditions allowing the multiplication of cells within the microcompartments, or the self-organization of cells into organoids;
the cell microcompartments are recovered.

36. The process according to claim 35, further comprising the step according to which:
the hydrogel layer is hydrolyzed to recover the organoids or cells.

37. The process as claimed in claim 35, wherein the cell microcompartments introduced contain pluripotent cells, said process comprising, inside the bioreactor, and a step of cell differentiation into at least one cell type of interest.

38. The process as claimed in claim 35, wherein the cell microcompartments introduced contain already differentiated cells or progenitors, said process comprising, inside the bioreactor, a step of multiplication or maturation of said differentiated cells in the microcompartments.

39. The process as claimed in claim 35, wherein the microcompartments introduced into the bioreactor have an initial cell density of less than 10% occupancy of the internal volume of the microcompartments.

40. The process as claimed in claim 35, wherein the microcompartments recovered at the end of the culture step in the bioreactor have a cell density greater than 10% occupancy of the internal volume of the microcompartments.

41. The process as claimed in claim 35, wherein the microcompartments introduced into the bioreactor have a cell density of less than 10% occupancy of the internal volume of said microcompartments, and the recovered microcompartments contain between 10% and 98% by volume of cells.
